(19) United States
(12) Patent Application Publication
(10) Pub. No.: US 2016/0045121 A1
(43) Pub. Date: Feb. 18, 2016

(54) System and Method for Monitoring Renal Sympathetic Nerve Activity

(71) Applicant: Indiana University Research and Technology Corporation, Indianapolis, IN (US)

(72) Inventors: A. George Akingba, Carmel, IN (US); Peng-Sheng Chen, Indianapolis, IN (US)

(21) Appl. No.: 14/823,346

(22) Filed: Aug. 11, 2015

Related U.S. Application Data
(60) Provisional application No. 62/036,433, filed on Aug. 12, 2014.

(51) Int. Cl. A61B 5/04; A61B 5/00; A61B 18/12; A61B 5/0205

(52) U.S. Cl. CPC A61B 5/04001; A61B 5/0205; A61B 5/4848; A61B 5/6876; A61B 5/6877; A61B 18/12; A61B 5/021

(57) ABSTRACT

A system and method for monitoring sympathetic nerve activity (SNA) includes obtaining nerve recordings from a subject using electrodes implanted to acquire signals from arteries or veins associated with sympathetic nerve activity of the subject and comparing the nerve recordings against at least one of pre-operative nerve recordings and post-operative nerve recordings to detect residual nerve activity of the subject.

### CROSS-REFERENCE TO RELATED APPLICATIONS

This application is based on, claims priority to, and incorporates herein by reference in its entirety U.S. Provisional Patent Application Ser. No. 62/036,433, filed Aug. 12, 2014, and entitled “SYSTEM AND METHOD FOR MONITORING RENAL SYMPATHETIC NERVE ACTIVITY.”

### STATEMENT REGARDING FEDERALLY SPONSORED RESEARCH

N/A

### BACKGROUND

The present disclosure relates to systems and methods for measuring electrical activity in nerves, and for applying electrical stimulation to nerves. More particularly, the disclosure relates to methods and electrodes for measuring renal sympathetic nerve activity (RSNA) and for applying electrical stimulation to the sympathetic nerves.

Many diagnostic and treatment methods in the fields of medicine and biology rely on measurements of nervous activity in patients and test subjects. Nervous activity in humans and other animals generates electrical signals that are detectable by electronic equipment such as oscilloscopes and other electrical signal processing devices. In order to detect the nerve activity, one or more electrical conductors, or electrodes, are placed in proximity to the nerves being measured. The electrodes may receive the electrical signals for further medical analysis. Various medical treatment methods also use electrodes to deliver electrical signals to the nerves in order to induce a response in the patient.

Renal care is one particular area of medical treatment that heavily utilizes measurement of nerve activity. Activity in the autonomic nervous system controls the variability of the heart rate and blood pressure. The sympathetic and parasympathetic branches of the autonomic nervous system modulate renal activity. Elevated levels of sympathetic nerve activity (SNA) are known to be correlated with heart failure, coronary artery disease, and may be associated with the initiation of hypertension. While elevated levels of SNA are known to be correlated with these medical conditions, more precise analysis of the particular electrical signals produced by sympathetic nerves is needed before sympathetic nerve measurement can become a useful diagnostic or prognostic tool. Deficiencies in current electrode technology result in either poor autonomic signal quality or present some difficulty in integrating implantable electronic enhancements (like telemetry, on-chip amplification, storage memory, and motion sensors).

One challenge to measuring sympathetic nerve activity is that the magnitude of electrical signals in the sympathetic nerves is relatively low, while various other electrical signals present in the patient provide noise that may interfere with isolation and detection of the sympathetic nerve activity. Existing electrodes detect both the nerve activity and other electrical noise generated in the patient's body. Thus, the signal to noise ratio (SNR) of the sympathetic nerve activity measured using electrodes known to the art is low, hindering the accurate detection and characterization of sympathetic nerve activity. For example, known electrodes have measured nerve signals with a voltage of 35 μV while the level of noise in the measuring electrode is 10 μV. Using the following SNR equation:

$$S N R = 20 log ( \frac{{V}_{signal}}{{V}_{noise}} ) dB ,$$

the example signals have an SNR of approximately 10.9 dB. While this signal to noise ratio permits some measurements of relatively large changes in sympathetic nerve activity, the noise level may mask nerve activity having a smaller voltage magnitude. Improvements to electrodes that increase the accuracy of nerve activity measurement, including sympathetic nerve activity measurement, will benefit the fields of medicine and biology.

Endovascular renal sympathetic denervation is an investigative therapy for resistant hypertension. It is necessary to monitor renal sympathetic nerve activity, particularly following sympathetic nerve ablation, which is an investigative therapy for managing resistant hypertension (HTN). Typically, the best way to confirm absent or diminished nerve activity after ablation is to get as close to the nerve as possible to measure the tiny changes in nerve potential that reflect true activity versus background cellular “noise.” Unfortunately, conventional ablation catheters do not get close enough to the nerve and typically demonstrate no difference in the primary outcome of blood pressure (BP) reduction. Thus, renal sympathetic nerve activity (RSNA) is not effectively abolished based on current techniques of renal nerve ablation, and thus their persists residual nerve activity post-ablation capable of sustaining the hypertensive state.

Further, most clinical endpoints following renal sympathetic nerve ablation are determined at a follow-up office visit that evaluates for BP reduction six months post-procedure. Other indices measured in such follow-ups include heart rate changes, epidermal growth factor receptor (eGFR), and epinephrine/norepinephrine level. In addition, no clinical trial has measured RSNA post-renal sympathetic nerve ablation. This creates uncertainty as to whether nerve ablation was complete or partial. In addition, one cannot know whether nerve regeneration occurs over time if RSNA is not measured chronically. Finally, the patho-physiological relationship between residual renal nerve activity post-ablation and durable BP reduction is not well demonstrated in animal or human subjects. In addition, although ablation technology already exists, a confirmatory step of monitoring nerve activity after the procedure has been absent from most clinical trials involving the conventional ablation catheters and similar devices.

Thus, there is a need enhance endovascular nerve monitoring, improve patient outcomes, and gain a greater understanding of systemic vascular hemodynamics. In addition, there is a need for improved systems and methods for evaluation of an intravascular nanotechnology-based device capable of facilitating continuous monitoring of renal sympathetic nerve activity—particularly following sympathetic nerve ablation.

### SUMMARY

The present disclosure recognizes that renal sympathetic nerve activity (RSNA) is not effectively abolished based on current techniques of renal nerve ablation, and thus there persists residual nerve activity post-ablation capable of sustaining the hypertensive state. Using an accessible endovascular approach, the present device is capable of placing a tiny monitor inside the renal artery, in proximity to the renal sympathetic nerves, in order to confirm nerve activity. This nerve-recording technique can more effectively evaluate whether renal nerve activity was abolished following endovascular ablation—which is desirable for decreasing blood pressure medications in patients with resistant hypertension.

The present disclosure provides a system that includes a planar nanoelectrode array (PNA) capable of obtaining high fidelity recordings of RSNA from an endovascular approach. Wire electrodes may be placed inside the renal artery as well as on the renal nerve located on the outside of the renal artery. The system includes a processor that obtains nerve recordings and validates the recorded nerve activity from inside a blood vessel alongside continuous blood pressure recordings utilizing the PNA. The processor may be configured to evaluate nerve activity pre-renal and post-renal nerve ablation to detect residual nerve activity post-ablation and its effects on blood pressure.

In some configurations, an electrode for measuring nerve activity has been developed. The electrode includes a first electrical contact having at least one electrically conductive projection extending from a surface of the first electrical contact, and a first electrical lead electrically connected to the first electrical contact to enable signals from the nerve to be received. The at least one electrically conductive projection is configured to engage tissue proximate to at least one nerve to enable the first electrical contact to electrically contact the nerve directly and form an electrically conductive or inductive path between the nerve and the electrical contact.

In one aspect of the present disclosure, a method is provided for measuring sympathetic nerve activity (SNA) that includes obtaining nerve recordings from a subject using electrodes implanted to acquire signals from arteries or veins associated with sympathetic nerve activity of the subject. The method also includes comparing the nerve recordings against at least one of pre-operative nerve recordings and post-operative nerve recordings to detect residual nerve activity of the subject and generating a report of residual nerve activity of the subject.

In another aspect of the present disclosure, a system is provided for monitoring sympathetic nerve activity (SNA). The system includes at least one electrode implanted to acquire signals from arteries or veins associated with SNA of a subject and a processor. The processor is configured to acquire feedback from the at least one electrode to assemble nerve recordings and compare the nerve recordings against at least one of pre-operative nerve recordings and post-operative nerve recordings to detect residual nerve activity of the subject.

The foregoing and other aspects and advantages of the disclosure will appear from the following description. In the description, reference is made to the accompanying drawings which form a part hereof, and in which there is shown by way of illustration a preferred embodiment of the disclosure. Such embodiment does not necessarily represent the full scope of the disclosure, however, and reference is made therefore to the claims and herein for interpreting the scope of the disclosure.

### BRIEF DESCRIPTION OF THE DRAWINGS

FIG. 1 is a diagram of sympathetic nerve fibers along a renal artery, pararenal aorta, and renal hilum.

FIG. 2 is a diagram of an ablation catheter advanced into the renal artery of FIG. 1.

FIG. 3A is a side view of a two-terminal nanoelectrode array in a silicon elastomer packaging.

FIG. 3B is a photograph of a two-terminal nanoelectrode array in a silicon elastomer packaging.

FIG. 3C is a cross-sectional view of the two-terminal nanoelectrode array of FIG. 3A and system for recording and analyzing/controlling the nanoelectrode array.

FIG. 4 an exemplary neurograph diagram showing an EKG recording, a SNA recorded by a conventional wire electrode, and a SNA recorded by planar nanoelectrode arrays (PNAs).

FIG. 5 is an exemplary neurograph diagram showing a chronic renal sympathetic nerve activity recording in a canine subject.

FIG. 6 is a flow chart setting forth steps of a method in accordance with the present disclosure.

### DETAILED DESCRIPTION

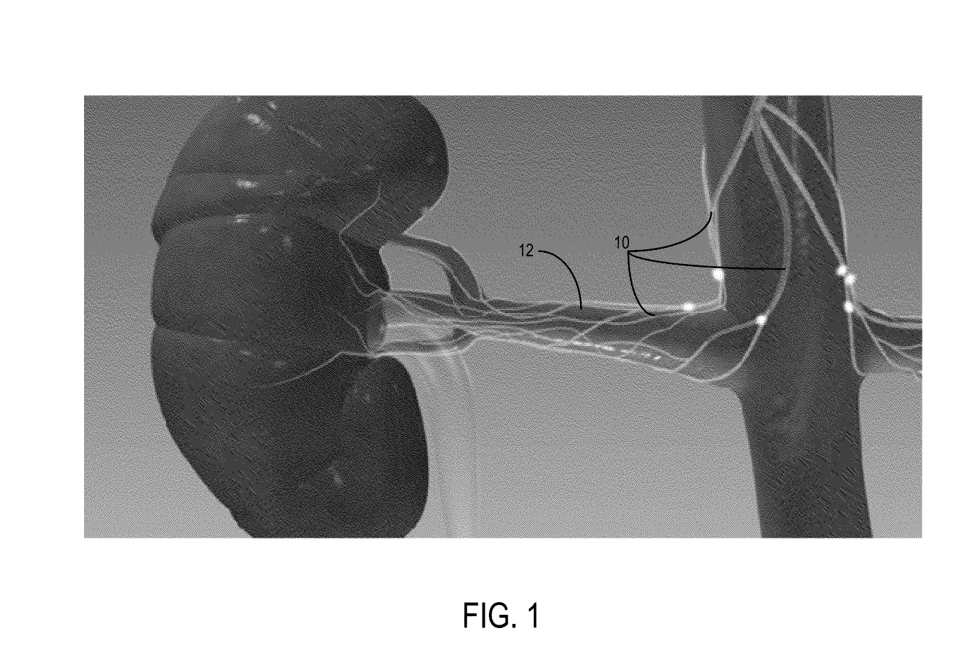

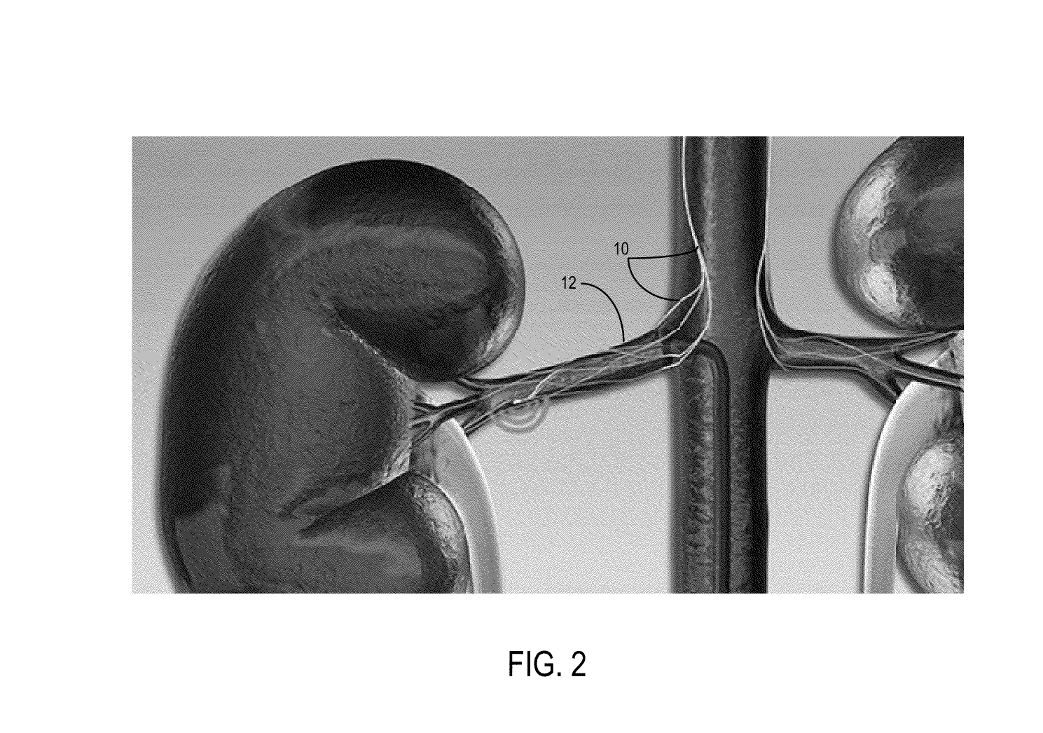

The sympathetic nervous system plays a key role in regulating human blood pressure, as evidenced by a profound improvement in blood pressure control after surgical sympathectomy. Endovascular renal sympathetic ablation (RSA) is a therapeutic approach for hypertensive patients who are not responsive to combination antihypertensive regimens (which may be 3 or more medications). As shown in FIGS. 1 and 2, the intent of endovascular ablation is to destroy the sympathetic nerve fibers 10 along both renal arteries 12, effectively blocking sympathetic activity. Response to ablative denervation should result in reduction or elimination of antihypertensive medications. However, with no way to directly measure lingering nerve activity, medications cannot be accurately adjusted and there is no way to tell if the procedure was successful. Thus, renal sympathetic nerve activity (RSNA) is not effectively abolished based on current techniques of renal sympathetic ablation (RSA).

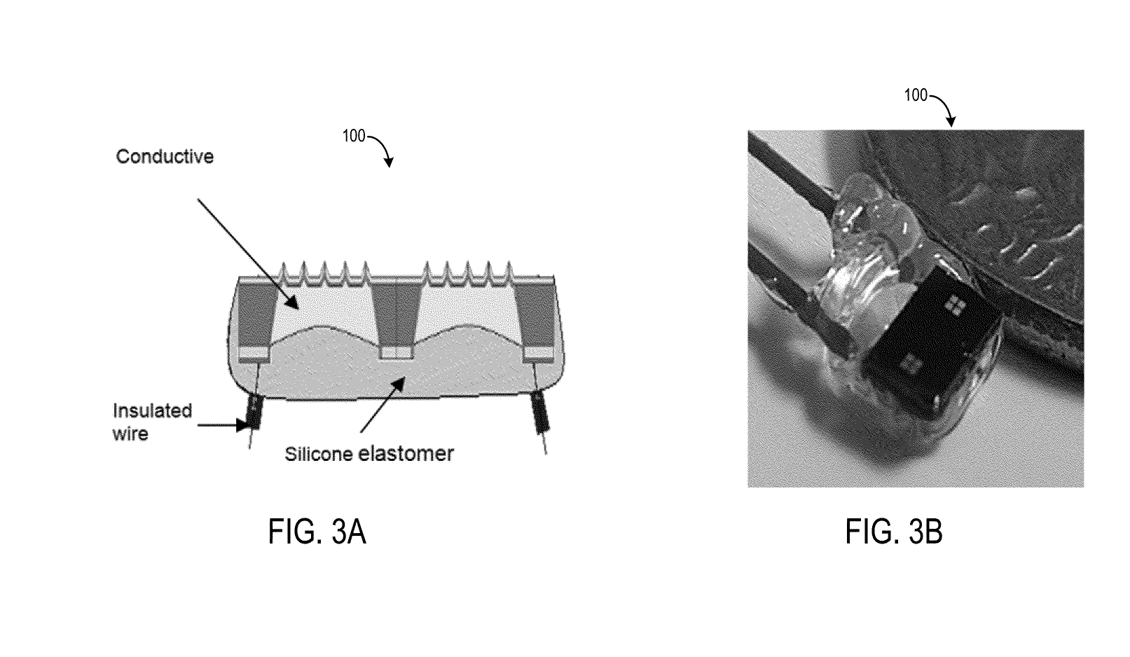

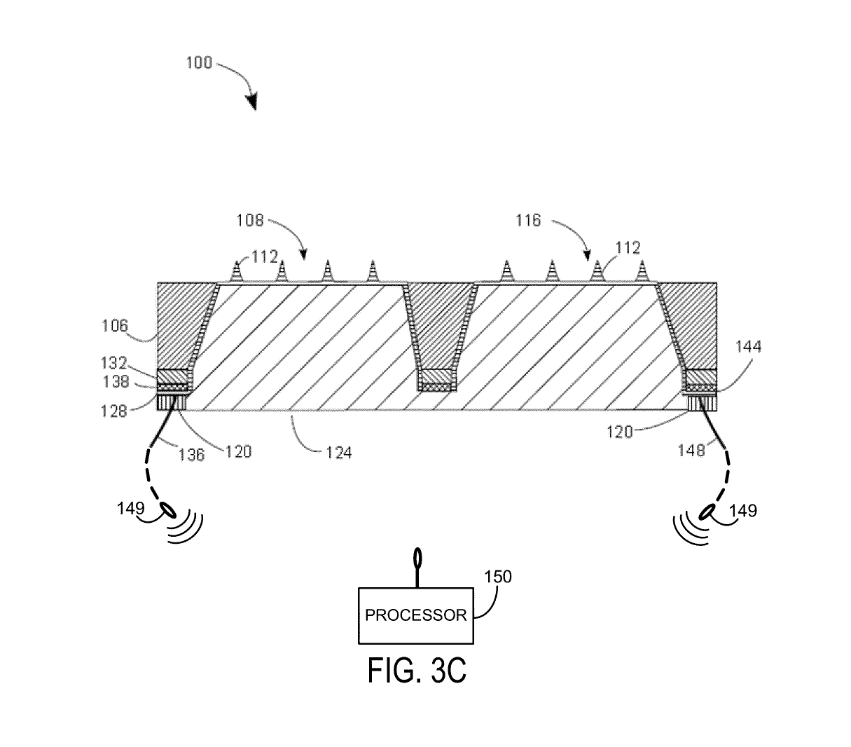

In addition, as previously discussed, using conventional wire electrode technology, the measurement of sympathetic nerve activity (SNA) has not been easy; for example, because of poor signal-to-noise ratio (SNR). However, the present disclosure provides a system and method where a nanoelectrode device 100, as shown in FIGS. 3A and 3B can be used to accurately detect and quantify residual activity. The nanoelectrode device 100 may be delivered endovascularly to measure residual nerve activity and thus evaluate the efficacy of renal sympathetic ablation. Further, the nanoelectrode device 100 may be used during an intraoperative, real-time nerve mapping procedure to more effectively navigate and target the optimal location for nerve ablation that may abolish nerve fiber activity in this area.

The nanoelectrode device 100 may include a bipolar nanoelectrode array capable of reducing electrode noise and improving signal-to-noise ratio. In addition to its increased surface contact (which boosts signal fidelity), the electrode may be sharp enough to penetrate the epineurium without damaging delicate sympathetic neurons. The nanoelectrode device 100 may have many clinical applications for directly detecting nerve activity that currently can only be indirectly measured. In one non-limiting example, the nanoelectrode device 100 may be a fully implantable electrode with no external wires that can deliver nerve activity data.

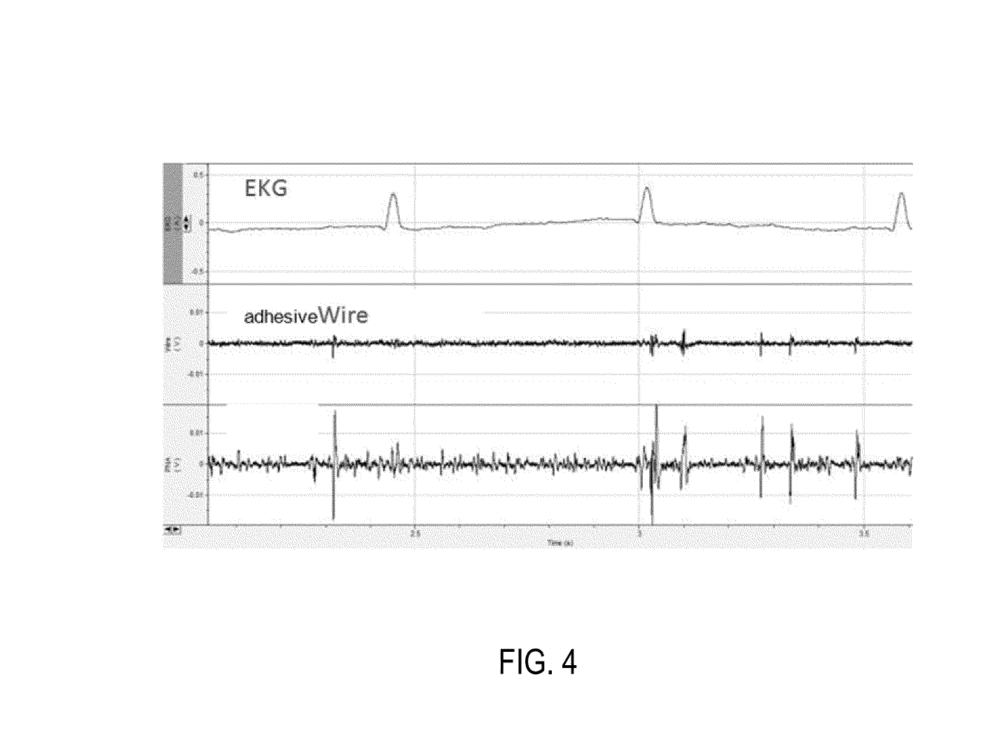

In another non-limiting example, the nanoelectrode device 100 may be a planar nanoelectrode array (PNA) device, as shown in FIGS. 3A and 3B, for measuring SNA. As shown in FIG. 4, improved SNR of SNA measurements may be achieved by the use of the nanoelectrode device 100. More specifically, FIG. 4 is an example neurograph diagram showing three channels recording the EKG, the SNA recorded by a conventional wire electrode, and a SNA recorded by PNAs. The diagram of FIG. 4 and other diagrams and reports may be generated by the processor 150 described below with respect to FIG. 3C. There is a 1-to-1 correspondence between the recorded events by both electrodes and it is qualitatively shown that the PNA device provides a better SNR. Also shown are smaller amplitude events that stand out as discernible events in the PNA device recordings but not with the wire electrodes.

In addition, nano-scale features on the nanoelectrode device 100 may also provide increased contact area with the host nerve. The nanoelectrode device 100 may be fabricated using standard complementary metal-oxide-semiconductor (CMOS) compatible techniques. The performance of the nanoelectrode device 100 may be compared with that of conventional wire electrodes by simultaneously measuring SNA from the same host nerve with both electrodes adjacent to each other on the stellate ganglion. The maximum recorded signal peak amplitudes were up to 167.25±27.17 μV for the PNA devices vs. 39.95±9.34 μV for the wire electrodes (p-value<0.001). Consequently there may be an improvement in the SNR of the measured SNA (36.59±1.51 dB for the PNA devices vs. 21.27±2.26 dB for the wire electrodes, (p-value<0.001)). Thus, the PNA devices may have a better SNR compared with wire electrodes. In addition, the PNA devices may be capable of recording signal events otherwise lost in the noise floor of the wire electrodes.

As best shown in FIG. 3C, the nanoelectrode device 100 includes two nanoelectrode arrays 108 and 116, electrically conductive layers 128 and 144, silicon layer 106, silicon oxide layer 132, dielectric layer 138, electrically conductive adhesive 120, electrically insulating adhesive 124, and electrical lead wires 136 and 148. The nanoelectrode arrays 108 and 116 may each include a plurality of nanoelectrode tips, such as tip 112, which extend from the surface of each electrode array. The silicon layer 106 and silicon oxide layer 132 electrically isolate the nanoelectrode arrays 108 and 116. The silicon layer 106 may be formed from a high resistivity silicon that resists a flow of electrical current between the electrically conductive layers 128 and 144. Each nanoelectrode tip 112 may be composed of gold or another electrical conductor that is fully exposed to contact nerve tissue for measuring nerve signals.

Each of the nanoelectrode arrays 108 and 116 can include an electrically conductive layer 128 and 144, respectively. The electrically conductive layers may include bonding pads for establishing an electrical connection to one of wires 136 and 148. The nanoelectrode arrays 108 and 116 and the electrical conductors are both formed from single layer of metal in some embodiments. In one embodiment, the electrically conductive layers 128 and 144 are formed from the same material as each nanoelectrode, such as gold, and promote a uniform electrical contact between each of the nanoelectrode tips and the electrical leads.

The electrically insulating adhesive 124 seals openings formed through the silicon layer 106, silicon oxide layer 132, and dielectric layer 138 to prevent fluids, tissue, or other contaminants from a patient or the environment surrounding the nanoelectrode device 100 from contacting the back side of either of the nanoelectrode arrays 108 and 116. In some configurations, the electrically insulative adhesive 124 does not completely fill the space under the nanoelectrode arrays 108 and 116, but seals an air pocket under each of the nanoelectrode arrays 108 and 106. Suitable adhesive materials include a silicone elastomer with a resistivity of 1.8×10¹⁵ Ω·cm and electrically insulative epoxies. One commercially available silicone elastomer is Dow Corning® 3745 RTV sold by the Dow Corning Corporation of Midland, Mich., USA. The electrically insulating adhesive 124, silicon layer 106, and silicon oxide layer 132 electrically isolate the nanoelectrode arrays 108 and 116. Thus, electrical nerve signals generated in the nerve tissue are conducted through the nanoelectrode arrays 108 and 116 through the leads 136 and 148, respectively, and do not form a circuit between the nanoelectrode arrays 108 and 116.

The electrical leads 136 and 148 may be formed from any electrically conductive material suited for use in a medical environment, including copper wires surrounded by an insulated jacket. Remote ends of wires 136 and 148 may connect to a variety of medical diagnostic equipment, for example, through wireless transmitters embedded 149 in the body of a patient. To this end, one general “processor” 150 is illustrated, which may be part of medical disagnositic equipment or a stand-along system. Additionally, the wires may connect to electrical signal generators for application of electrical stimulation to various nerves.

In operation, the nanoelectrode device 100 is placed in contact with tissue of a patient or test subject proximate to a nerve undergoing measurement or electrical stimulation. The nanoelectrode tips in nanoelectrode arrays 108 and 116 can detect electrical signals in the nerve tissue in two different ways. First, the nanoelectrode tips penetrate a layer of tissue that is proximate to a nerve while not damaging the nerve.

Both of the nanoelectrode arrays 108 and 116 may penetrate the surrounding tissue and establish low electrical resistance contact with nerve cells. Second, the nanoelectrode arrays 108 and 116 can detect electrical signals through induction. The electrical activity in the nerve tissue generates an electrical field that induces a current in each of the nanoelectrode arrays. In the two-terminal configuration of nanoelectrode device 100, each of the nanoelectrode arrays 108 and 116 detects a separate electrical signal in the nervous tissue. A differential amplifier, such as a differential operational amplifier or other detector, generates a signal corresponding to a difference between the voltages generated in each of the nanoelectrode arrays 108 and 116 for use with signal detection and medical diagnostic equipment.

A system that measures electrical activity in nervous tissue of a patient can also detect spurious electrical signals, referred to as noise, from other sources than the nerve tissue. Sources of noise include diagnostic equipment connected to the terminals and external electromagnetic signals that generate noise in the electrical leads attached to the terminals. Various techniques known to the art can mitigate some external sources of noise. One source of noise, referred to as the Johnson noise also called Johnson-Nyquist noise, Nyquist noise or thermal noise, is electronic noise generated by the thermal agitation of charge carriers in an electric conductor and occurs regardless of any applied voltage. The Johnson noise level in an electrical circuit can be expressed as a voltage Vⱼ and is expressed with the following equation:

Vⱼ=\sqrt{4k_{b}RTΔf}

Where k_{b} is Boltzmann's constant, R is the resistance of the circuit (including and dominated by the resistance of the electrodes inserted for measurements in applications like this one) in Ohms, Δf is the frequency bandwidth in Hz of the signal at the terminal and T is the temperature in degrees Kelvin. In a practical situation, the body temperature of a patient provides the temperature T. Additionally, narrowing frequency bandwidth Δf can reduce noise, but leads to a loss of information in the nerve signal being measured by the nanoelectrode. The nanoelectrode terminals in the nanoelectrode device 100 establish electrical contacts with nerves over a broad surface area that have a lower electrical resistance between the nerves and the electrode than electrodes previously known in the art. The reduction of the resistance R also reduces the magnitude of noise voltage Vⱼ and consequently reduces measured noise when measuring nerve activity, without narrowing the frequency bandwidth Δf. The reduction in noise results in improved signal to noise ratios when measuring nerve activity, including sympathetic nerve activity. Thus, the structure of the terminals in the nanoelectrode device 100 enable improved detection of electrical nervous activity over prior art devices.

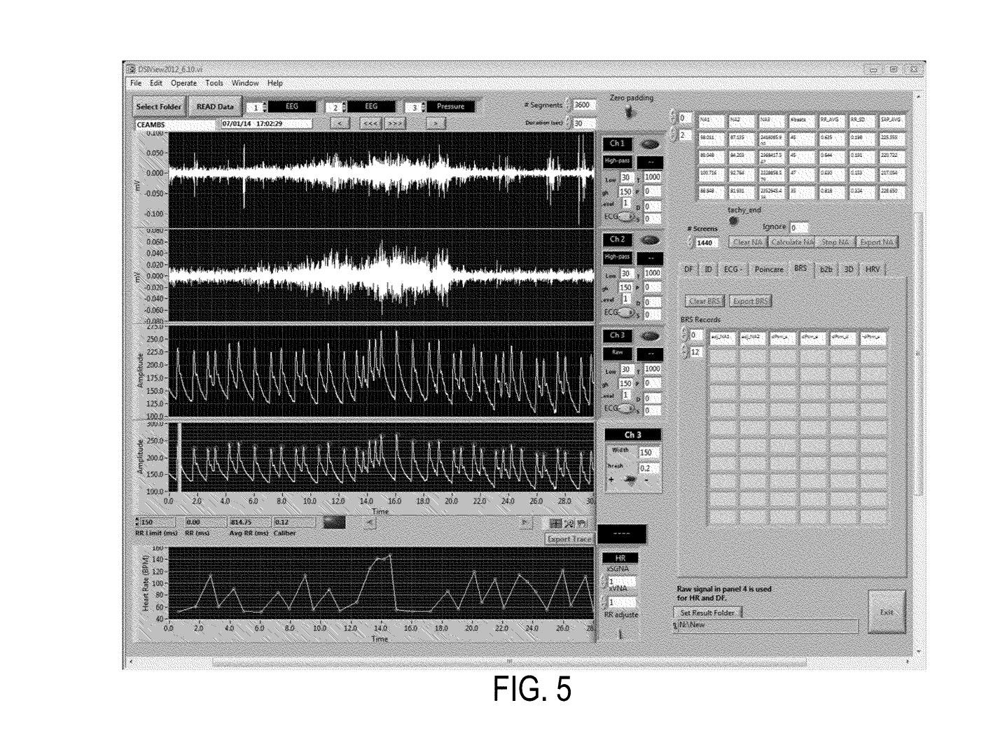

Using an open surgical technique, an intravascular blood pressure probe and wire electrodes for nerve activity recording may be implanted, both intravascular and extravascular to the renal artery, to simultaneously obtain baseline RSNA data. The wire electrode placed intravascularly may be secured close to the vessel wall to acquire the nerve activity. Successful nerve recording data obtained from this approach, such as the data shown in FIG. 5, may validate the ability to obtain nerve activity from inside a blood vessel and evaluate the relationship between recorded nerve activity and blood pressure changes effected by using vasodilator and vasoconstrictor medications. The report of FIG. 5 and other diagrams and reports may be generated by the processor 150 described above with respect to FIG. 3C. Long-term changes in intra-arterial BP of the subject during daily activities (e.g., whether asleep or awake) may be measured and cyclical BP changes with RSNA may be correlated. In addition, renal RSNA, BP, and serum marker responses to infusions of medications that regulate blood pressure may be measured.

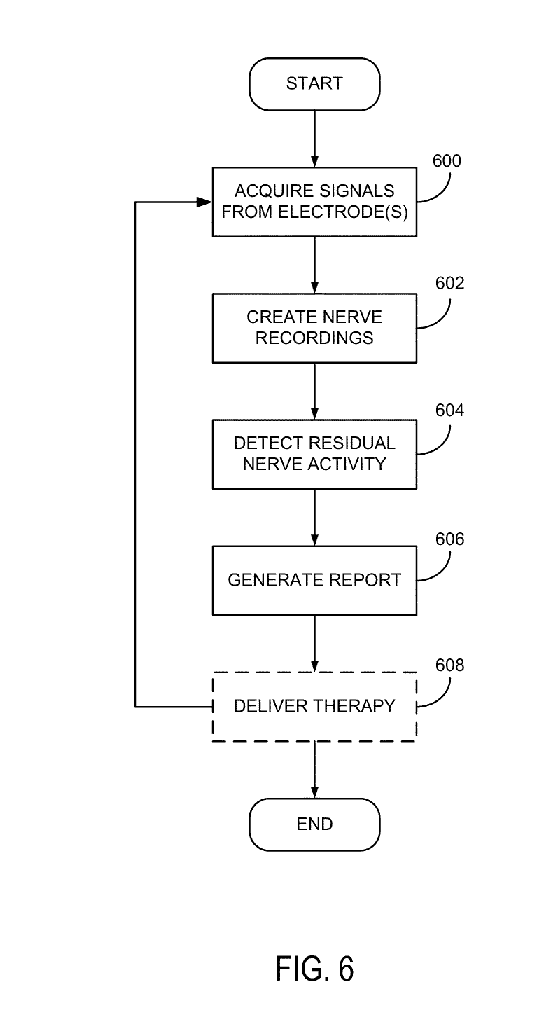

Referring to FIG. 6, a process for monitoring sympathetic nerve activity (SNA), such as renal sympathetic nerve activity (RSNA) may begin at process block 600 with the acquisition of signals from electrodes, such as described above. At process block 602, a system or processor such as described above may assemble the signals into nerve recordings. The nerve recordings may then be compared to reference information or other recordings, such as pre-operative nerve recordings and/or post-operative nerve recordings. Thus, at process block 604, residual nerve activity of the subject may be detected. At process block 606 a report, for example, indicating the residual nerve activity that was detected. The report may be electronic and displayed. In electronic format, the report may be a communicated signal, such as may be received by a therapy device whereby, at optional process block 608, a therapy may be triggered. The process may continue or end.

Various hardware and systems may be used to implement the above-described processes. In one non-limiting example, three probes may be implanted in the renal artery and pararenal aorta. After exposing the retroperitoneum and renal hilum, a first probe (e.g., electrode for nerve activity measurement) may be placed inside one renal artery. Fluoroscopy techniques may be used to confirm accurate placement of the first probe. Next, a second probe (e.g., probe for blood pressure measurement) may be placed in the infrarenal aorta or renal artery, followed by a third probe placed on the ipsilateral renal sympathetic nerve (RSN), located on the outside part of renal artery.

After allowing one to two weeks for the subjects to heal sufficiently to obtain stable readings, data on continuous nerve activity, continuous BP, heart rate, and locomotor activity may be obtained for approximately two weeks. Next, an intravenous catheter may be placed in each subject. In one non-limiting example, one dose of 100 mcg/kg phenylephrine may be given to each subject via IV route. Blood pressure should rise to 120-140 mm Hg. If the blood pressure does not rise to 120-140 mm Hg, phenylephrine 1 mg/mL may be slowly inserted at a rate of 0.5-1 mm Hg/s until a reading of 120-140 mm Hg is achieved. Approximately 30-60 minutes later, hemodynamic parameters should return to baseline. Next, one dose of 2.5 mcg/kg sodium nitroprusside via IV route may be given to the subject to exhibit a blood pressure drop of about 20-40 mm Hg. However, if the blood pressure drop of about 20-40 mm Hg does not occur, 1 mg/mL sodium nitroprusside may slowly be instated into the subject at a rate of 0.5-1 mmHg/s. The nerve activity, BP, heart rate, and locomotor activity may continuously be recorded.

In another non-limiting example, traditional electrodes, as just described, may be placed both intravascular and extravascular to the renal artery in addition to catheter-directed renal nerve ablation. Therefore, the relationship between recorded nerve activities and blood pressure changes post-ablation of the renal nerves may be evaluated to identify residual nerve activity post-RSN ablation and its post-ablative effects on BP modulation.

Once data is collected on continuous nerve activity, continuous BP, heart rate, and locomotor activity, as previously described, conscious sedation and general anesthesia of the subjects may be compared and the contralateral common femoral artery may be accessed. A conventional radiofrequency ablation (RFA) catheter, such as the Medtronic Simplicity RFA catheter, may be used for bilateral renal sympathetic ablation by IFU protocol: 8 Watts (5-8 Watts), 4-6 times, each ablation for 120 s, moving from distal to proximal, spacing 5 mm each treatment interval. Continuous recording of all indices may then be completed. In addition, perioperative blood samples may be obtained to measure representative biochemical biomarkers (e.g., epinephrine and norepinephrine).

In yet another non-limiting example, nerve activity may be recorded by traditional wire electrodes and the nanoelectrode device 100 pre-renal and post-renal nerve ablation. In this example, both traditional and the nanoelectrode device 100 are placed within the renal arteries as previously described. Then catheter-directed renal nerve ablation may be performed and continuous monitoring recorded. Again, the relationship between recorded nerve activities and blood pressure changes pre-ablation and post-ablation may be evaluated. However, in this stage, the quality of the recordings may be compared between the traditional wire electrode and the nanoelectrode device 100. The signal-to-noise ratio of each type of electrode may also be analyzed and compared, with the hypothesis that the nanoarray electrode will be better able to discriminate between cellular “noise” and nerve activity.

In some embodiments, the nanoelectrode device 100 may be a minimally invasive diagnostic and therapeutic device that can be delivered via an endovascular route to be implanted long-term in an area where direct monitoring has heretofore not been possible. This may expand the clinical information to be gathered and allow for continuous measurements that current monitors located outside of the body are not capable of conveniently doing. The nanoelectrode device 100 may further be used in managing patients with wireless implanted devices capable of monitoring therapeutic procedures in ways that minimize or eliminate frequent imaging, lab draws or prolonged percutaneous catheterizations.

In other embodiments, the nanoelectrode device 100 may be monolithically integrated with amplifiers and wireless transceivers for seamless telemetry that can be delivered through an endovascular approach. This will allow users to dispense with transcutaneous electrodes and their accompanying complications. The nanoelectrode device 100 may also be used to evaluate other visceral disorders under autonomic control (e.g., gastrointestinal dysmotility, urinary bladder dysfunction, erectile or ejaculatory dysfunction).

The present invention has been described in terms of one or more preferred embodiments, and it should be appreciated that many equivalents, alternatives, variations, and modifications, aside from those expressly stated, are possible and within the scope of the invention.

1. A method for measuring sympathetic nerve activity (SNA) comprising:
obtaining nerve recordings from a subject using electrodes implanted to acquire signals from arteries or veins associated with sympathetic nerve activity of the subject;
comparing the nerve recordings against at least one of pre-operative nerve recordings and post-operative nerve recordings to detect residual nerve activity of the subject; and
generating a report of residual nerve activity of the subject.

2. The method of claim 1 wherein the nerve recordings are acquired after a nerve ablation procedure and wherein the residual nerve activity is relative to the nerve targeted by the nerve ablation procedure.

3. The method of claim 1 wherein the arteries are the renal arteries and the veins are renal veins and the SNA is renal SNA (RSNA).

4. The method of claim 1 wherein the electrodes form a planar nanoelectrode array (PNA) configured to obtain high fidelity recordings of renal SNA (RSNA) from an endovascular deployment.

5. The method of claim 1 wherein the electrodes include wire electrodes placed inside one of a renal artery or a renal vein and on the renal nerve located outside the one of the renal artery or the renal vein.

6. The method of claim 1 further comprising evaluating a relationship between the nerve recordings and blood pressure changes effected by using vasodilator and vasoconstrictor medications.

7. The method of claim 1 further comprising monitoring changes in intra-arterial or intra-venous blood pressure of the subject during daily activities and cyclical blood pressure changes with SNA and indicating a correlation in the report.

8. The method of claim 1 further comprising performing a therapy based on the report.

9. The method of claim 8 wherein the report is an electronic report received by an electronic device configured to perform the therapy.

10. A system for monitoring sympathetic nerve activity (SNA) comprising:
at least one electrode implanted to acquire signals from arteries or veins associated with SNA of a subject;
a processor configured to:
acquire feedback from the at least one electrode to assemble nerve recordings;
compare the nerve recordings against at least one of pre-operative nerve recordings and post-operative nerve recordings to detect residual nerve activity of the subject.

11. The system of claim 10 wherein the processor is further configured to determine the residual nerve activity relative a nerve targeted by a nerve ablation procedure.

12. The system of claim 10 wherein the electrodes form a planar nanoelectrode array (PNA) configured to obtain high fidelity recordings of renal SNA (RSNA) from an endovascular deployment.

13. The system of claim 10 wherein the electrodes include wire electrodes placed inside a renal artery or a renal vein and on the renal nerve located outside the renal artery or the renal vein.

14. The system of claim 13 wherein the SNA is renal SNA (RSNA).

15. The system of claim 10 wherein the processor is further configured to evaluate a relationship between the nerve recordings and blood pressure changes effected by using vasodilator and vasoconstrictor medications.

16. The system of claim 10 wherein the processor is further configured to generate a report of residual nerve activity of the subject.

17. The system of claim 16 wherein the processor is further configured to monitor changes in intra-arterial or intra-venous blood pressure of the subject during daily activities and cyclical blood pressure changes with SNA and indicating a correlation in the report.

18. The system of claim 16 wherein the processor is further configured to trigger performance of a therapy using the report.
